(19) United States
(12) Patent Application Publication
(10) Pub. No.: US 2021/0085591 A1
(43) Pub. Date: Mar. 25, 2021

(54) COSMETIC

(71) Applicant: SHISEIDO COMPANY, LTD., TOKYO (JP)

(72) Inventors: Atsushi SOGABE, YOKOHAMA-SHI KANAGAWA (JP); Ayano MATSUO, YOKOHAMA-SHI KANAGAWA (JP)

(73) Assignee: SHISEIDO COMPANY, LTD., TOKYO (JP)

(21) Appl. No.: 16/954,504

(22) PCT Filed: Dec. 27, 2018
(86) PCT No.: PCT/JP2018/048064

§ 371 Date: Jun. 16, 2020

(30) Foreign Application Priority Data
Dec. 28, 2017 (JP) 2017-254460

(51) Int. Cl. A61K 8/81; C08F 20/06; A61K 8/06; A61Q 19/00; A61K 8/891; A61K 8/37; A61K 8/73

(52) U.S. Cl. CPC A61K 8/8147; C08F 20/06; A61K 8/062; A61K 8/73; A61K 8/891; A61K 8/37; A61Q 19/00

(57) ABSTRACT

A cosmetic includes a component (a) and (b): wherein component (a) is a polyacrylic acid or a salt thereof or poly(2-acrylamido-2-methylpropanesulfonic acid) or a salt thereof that has a weight-average molecular weight of 500,000 to 8,000,000, is linear, and has a thread length of 10 mm or less at room temperature when formed into a 1% by mass solution, wherein the thread length is a distance determined by: bringing a round disk having a diameter of about 1 cm uniformly and lightly into contact with a surface of the solution; lowering a container that contains the solution at a velocity of 5 mm/sec, and measuring the distance that the container descended until a thread of the solution is cut, and component (b) is a crosslinked water-swellable polymer having a crosslinking density of 0.01 to 1% by mole, or a microgel obtained by pulverizing a gel consisting of a hydrophilic compound having gelating ability.

### RELATED APPLICATION

The present invention is based upon and claims the benefit of the priority of Japanese Patent Application No. 2017-254460 (filed on Dec. 28, 2017), the disclosure of which is incorporated herein in its entirety by reference.

### FIELD OF THE INVENTION

The present invention relates to a cosmetic, and more specifically to a cosmetic blended with a precisely synthesized polymer.

### BACKGROUND OF THE INVENTION

Freshness, good spreadability, and non-stickiness are properties desired to many cosmetics, but it is not easy to exhibit rich feeling and full-bodied feeling without impairing these properties.

It is generally known that rich feeling of cosmetics correlates with the viscoelastic ratio thereof, and full-bodied feeling can be evaluated with the gradient of the first normal stress difference (Patent Literature 1).

The viscoelastic ratio and the gradient of the first normal stress difference of cosmetics greatly depend on blending of thickeners. Examples of thickeners commonly used in cosmetics include anionic polymers and polysaccharides. When anionic polymers absorb water, they gelate and exhibit a thickening effect; however, it is difficult to exhibit rich feeling and full-bodied feeling when blended in large amounts and, stringiness (thread-forming property), which is undesirable for cosmetics, tends occur. Moreover, polysaccharides have a problem of causing stickiness when blended in large amounts since they do not penetrate the skin and remain on the skin.

Under such circumstances, a component that does not have stringiness and is capable of imparting rich feeling and full-bodied feeling without impairing freshness, good spreadability, and non-stickiness of a cosmetic was desired.

### CITATION LIST

### Patent Literature

Patent Literature 1: Japanese Unexamined Patent Publication No. 2013-137266 A.

Patent Literature 2: International Publication No. WO2015/052804 A

Patent Literature 3: Japanese Patent No. 5076428 B.

### DISCLOSURE OF THE INVENTION

### Problem to be Solved by the Invention

The present invention was made in view of the above-described problems in conventional art, and an object of the present invention is to provide a cosmetic that is excellent in rich feeling and full-bodied feeling, does not have stringiness, and is also excellent in freshness, spreadability, and non-stickiness.

### Means to Solve the Problem

The present inventors have been working on the development of thickeners that are suitable for cosmetics, and have reported that “polyacrylic acid or a salt thereof or poly(2-acrylamido-2-methylpropanesulfonic acid) or a salt thereof that has the weight-average molecular weight of 500,000 to 8,000,000, has the content of molecular species having the molecular weight of 10,000,000 or more of 10% by mass or less; and is linear” can be used as a thickener capable of providing a thickening effect to cosmetics without imparting stringiness (Patent Literature 2). It is described that this effect can be obtained because the stringiness is significantly reduced with the “polyacrylic acid or a salt thereof or poly(2-acrylamido-2-methylpropanesulfonic acid) or a salt thereof” than the compound of a conventional one (Patent Literature 2).

The present inventors have diligently studied the above-described problems. As a result, they have found that when the “polyacrylic acid or a salt thereof or poly(2-acrylamido-2-methylpropanesulfonic acid) or a salt thereof” is added to an aqueous phase in which a commonly used thickener is dissolved, the viscoelastic ratio and the gradient of the first linear stress difference increase synergistically, and significant rich feeling and full-bodied feeling can be exhibited. Moreover, the present inventors have found that a cosmetic prepared by using said thickener in combination has excellent rich feeling and full-bodied feeling, does not have stringiness, and also has excellent freshness, spreadability, and non-stickiness, and thus completed the present invention.

That is, the present invention encompasses the following.

[1] A cosmetic comprising components (a) and (b):

(a) polyacrylic acid or a salt thereof or poly(2-acrylamido-2-methylpropanesulfonic acid) or a salt thereof that has a weight-average molecular weight of 500,000 to 8,000,000, is linear, and has a thread length of 10 mm or less at room temperature when formed into a 1% by mass solution, wherein the thread length is a distance determined by: putting a round disk having a diameter of about 1 cm uniformly and lightly into contact with a surface of the solution; lowering a container that contains the solution at a velocity of 5 mm/sec, and measuring the distance that the container descended until a thread of the solution is cut, and

(b) a crosslinked water-swellable polymer having a crosslinking density of 0.01 to 1 mol %, or a microgel obtained by pulverizing a gel consisting of a hydrophilic compound having gelation ability.

[2] The cosmetic according to [1], wherein the crosslinked water-swellable polymer of the component (b) is one or more selected from the group consisting of a carboxyvinyl polymer, acrylamidealkylsulfonic acid/beheneth-25 crosslinked copolymer, acrylamidealkylsulfonic acid/vinylpyrrolidone crosslinked copolymer and acrylamidealkylsulfonic acid/alkylacrylamide crosslinked copolymer.
[3] The cosmetic according to [1] or [2], wherein the microgel of the component (b) is a hydrophilic polysaccharide.
[4] The cosmetic according to any one of [1] to [3], wherein the content of the compound having the molecular weight of 10,000,000 or more in the component (a) is 10% by mass or less.

### Effect of the Invention

The present invention provides a cosmetic that is excellent in rich feeling and full-bodied feeling, does not have stringiness, and also excellent in freshness, spreadability, and non-stickiness.

### BRIEF DESCRIPTION OF THE DRAWING

FIG. 1 is a graph showing the results of analyzing the viscoelastic ratio, which is an index of rich feeling, and the gradient of the first normal stress difference, which is an index of full-bodied feeling, of the thickeners used in the Examples of the present application.

### BEST MODE FOR CARRYING OUT THE INVENTION

Preferred embodiments of the present invention are described in the following.

Herein, the “linear polymer having a weight-average molecular weight of 500,000 to 8,000,000 and a thread length of 10 mm or less at room temperature when formed into a 1% by mass solution” may be referred to as a “precisely synthesized polymer”. The “thread length” is a value measured by the method described in Patent Literature 2. Namely, a value defined as “a distance determined by putting a round disk having a diameter of about 1 cm uniformly and lightly into contact with a surface of the solution, lowering a container at the velocity of 5 mm/sec, and measuring the distance that the container descended until a thread of the solution is cut”. In the “linear polymer having the weight-average molecular weight of 500,000 to 8,000,000” that satisfies the requirement of the thread length, the “content of molecular species having the molecular weight of 10,000,000 or more” is usually “10% by mass or less”, and thus the requirement of “the content of molecular species having a molecular weight of 10,000,000 or more is 10% by mass or less” may be optional.

Polyacrylic acid and poly(2-acrylamido-2-methylpropanesulfonic acid) having the properties of the precisely synthesized polymer may be referred to as “precisely synthesized polyacrylic acid” and “precisely synthesized PAMPS,” respectively. Here, “PAMPS” is an abbreviation for poly(2-acrylamido-2-methylpropanesulfonic acid).

Moreover, in the precisely synthesized polymer, the content of the compound having the molecular weight that is three times or more of the weight-average molecular weight may be 10% by mass or less. This is because the stringiness of the precisely synthesized polymer tends to be reduced more.

[Component (a)]

In the present invention, polyacrylic acid or a salt thereof or poly(2-acrylamido-2-methylpropanesulfonic acid) or a salt thereof that has a weight-average molecular weight of 500,000 to 8,000,000, has a thread length of 10 mm or less at room temperature when formed into a 1% by mass solution, and is linear can be used as the component (a). Namely, the precisely synthesized polyacrylic acid or a salt thereof or the precisely synthesized PAMPS or a salt thereof can be used as the component (a).

Examples of the kinds of the above-described salts include alkali metal salts (for example, sodium salt, potassium salt, magnesium salt, calcium salt, etc.), organic amine salts (for example, monoethanolamine salt, diethanolamine salt, triethanolamine salt, triisopropanolamine salt, etc.), and the salts of basic nitrogen-containing compounds such as 2-amino-2-methyl-1-propanol, 2-amino-2-methyl-1,3-propanediol, 2-amino-2-hydroxymethyl-1,3-propanediol, L-arginine, L-lysine, and L-alkyl taurine, etc. Among them, monovalent alkali metal salts and organic amine salts are preferred, a sodium salt, potassium salt and triethanolamine salt are more preferred, and a sodium salt is the most preferred.

In the present invention, the polyacrylic acid salt or PAMPS salt indicates a compound obtained by neutralizing the polyacrylic acid or PAMPS with the above-described base (namely, the above-described alkali metals, organic amines, basic nitrogen-containing compounds, etc.) or a compound obtained by polymerizing the acrylic acid or 2-acrylamide-2-methylpropanesulfonic acid (hereinafter abbreviated as AMPS) whose acid section is neutralized in advance with the above-described base.

As the examples of the precisely synthesized polymers, those that can be synthesized by a RAFT polymerization method to be described later are preferable. Examples include homopolymers and/or salts thereof in which acrylic acid monomers such as methacrylic acid, alkyl acrylates, alkyl methacrylates, acrylic acid esters; acrylamide monomers such as acrylamide and dimethylacrylamide; vinyl monomers such as vinyl alcohol, vinylpyrrolidone, vinyl acetate, carboxyvinyl, and vinyl methyl ether; and styrene, urethane, etc. are the constituent unit monomer; and copolymers and/or salts thereof consisting of two kinds or more of the monomers selected from these monomers, acrylic acid, and AMPS. Among them, those in which the constituent unit is an acrylic acid monomer or acrylamide monomer are especially preferable. Furthermore, macro-monomers in which polyethylene glycol, silicone-based polymer compound, etc. are added to the above-described monomer, as the side chain, can also suitably be used as the constituent unit.

Specific examples of the compound include polyacrylamide, polydimethylacrylamide, polyvinyl alcohol, polyvinylpyrrolidone, polyvinylmethyl ether, polyvinyl acetate, carboxyvinyl polymer, etc.; and (acrylic acid/alkyl acrylate) copolymer, (acrylic acid/alkyl methacrylate) copolymer, (alkyl acrylate/styrene) copolymer, polyacrylic acid ester copolymer, (dimethylacrylamide/2-acrylamido-2-methylpropanesulfonic acid) copolymer and salts thereof.

The amount of the component (a) blended in the cosmetic according to the present invention is 0.005 to 2% by mass, preferably 0.005 to 1.5% by mass, and more preferably 0.005 to 1% by mass. When the blending amount is less than 0.005% by mass, a sufficient normal stress may not be achieved, and when the blending amount exceeds 2% by mass, the normal stress may be too high and cause a slimy texture.

Method for Synthesizing Precisely Synthesized Polymer

The precisely synthesized polymer of the present invention can be synthesized by a publicly known living polymerization method. Examples of living polymerization include living anionic polymerization, living cationic polymerization, living radical polymerization (precise radical polymerization, or controlled radical polymerization), etc.

Examples of living radical polymerization include (radical) polymerization that is mediated by nitroxide, or nitroxide-mediated (radical) polymerization (NLRP), atom transfer radical polymerization (ATRP), reversible addition-fragmentation chain transfer (RAFT) polymerization, etc. Examples of atom transfer radical polymerization (ATRP) include electron transfer generated activator ATRP, or activators generated by electron transfer ATRP (AGET ATRP), electron transfer regenerated activator ATRP, or activators regenerated by electron transfer ATRP (ARGET ATRP), initiators to continuously regenerate active species ATRP, or initiators for continuous activator regeneration ATRP (ICAR ATRP), and reverse ATRP (Reverse ATRP). The RAFT polymerization method is a living radical polymerization method that uses a RAFT agent as a chain transfer agent. Examples of a derivative technology of the RAFT polymerization include living radical polymerization in which organic tellurium is the growing end, or organic tellurium-mediated living radical polymerization (TERP), antimony-mediated living radical polymerization (SBRP), and bismuth-mediated living radical polymerization (BIRP), etc. Examples of other living radical polymerizations include iodine transfer radical polymerization (IRP), cobalt-mediated radical polymerization (CMRP), etc.

The direct polymerization of acrylic acid is preferable because of the simplicity of polymerization. However, when the polymerization is difficult because of the formation of insoluble salts such as catalysts, protected acrylic acid esters such as t-butyl acrylate, methoxymethyl acrylate, and methyl acrylate are used, and the desired polymer compound can be obtained by the succeeding deprotection.

In the present invention, the living radical polymerization method is preferable because the precision synthesis of a high molecular weight body (namely, the synthesis of polymer compounds with narrow molecular weight distribution) is possible, and the reversible addition-fragmentation chain transfer polymerization method (RAFT polymerization method) is more preferable (Patent Literature 2).

It is also known that secondary reactions such as branching and crosslinking easily occur in other polymerization methods, but branching and crosslinking does not occur easily in the living radical polymerization method.

In particular, the RAFT polymerization method is a polymerization method in which growth continues while exchanging a chain transfer agent (a RAFT agent) between active polymer ends; namely, living is achieved by a so-called exchange chain mechanism. When the RAFT agent bonds to a growth end of the polymer chain, it becomes a resting state (dormant state); and when the RAFT agent is detached, a growth reaction occurs. However, because the equilibrium state of bonding is significantly biased toward the bonding side (i.e., the time when the RAFT agent is bonded is much longer than the time when the RAFT agent is detached), the growth rate of the polymer chain is very slow, and the reactivity of the end can be suppressed. Accordingly, since the rate of the growing reaction in each polymer chain becomes the same, and the degree of polymerization of the polymer is basically proportional to the reaction time, a polymer having an extremely narrow molecular weight distribution can be obtained. Moreover, since the reactivity is low, it is considered that secondary reactions such as branching and crosslinking does not occur easily.

A dithiocarbonyl compound and a trithiocarbonyl compound can be preferably used as RAFT agents (i.e., chain transfer agents). Dithiocarbamate and trithiocarbamate are more preferred, and 4-cyanopentanoic acid dithiobenzoate and α-(methyltrithiocarbonate)-S-phenylacetic acid are most preferred. The polymerization initiator that has a similar chemical structure as that of the chain transfer agent is preferred, and an azo initiator is preferred. The polymerization solvent is not limited in particular, and those having high solubility of monomers and polymers are suitably selected. The polymerization time is preferably several hours to 100 hours.

Method for Measuring Molecular Weight

The molecular weight of the precisely synthesized polymer can be measured by publicly known methods, such as a light scattering method, an ultracentrifugal method, and a chromatographic method for the weight-average molecular weight; and an osmometric method and a chromatographic method for the number-average molecular weight. Among them, the chromatographic method is preferred because the weight-average molecular weight, the number-average molecular weight, and the molecular weight distribution can easily be obtained with a small amount of sample; in particular, a gel permeation chromatographic method (hereinafter abbreviated as GPC) is preferred.

The molecular weight distribution used in this application is the value determined by dividing the weight-average molecular weight obtained by GPC analysis by the number-average molecular weight.

[Component (b)]

In the present invention, (b) a crosslinked water-swellable polymer having a crosslinking density of 0.01 to 1 mol %, or a microgel obtained by crushing a gel consisting of a hydrophilic compound having gelation ability can be used as the component (b).

The crosslinked water-swellable polymer may be a polymer based on (meth)acrylic acid or modified (meth)acrylic acid. Examples thereof include, but not limited to: crosslinked polymers of acrylic acid represented by a carboxyvinyl polymer (a carbomer), copolymers of (meth)acrylic acid and polyalkylene polyether, hydrophobically modified poly(meth)acrylates, (meth)acrylate/C10-30 alkyl acrylate polymers, (meth)acrylates/beheneth-25 methacrylate copolymers, (meth)acrylate/(meth)acrylamide copolymers, (meth)acrylate/(meth)alkylacrylamide copolymers, (meth)acrylate/(meth)hydroxyethylacrylamide copolymers, and (meth)acrylate/polyalkyleneoxide alkyl-modified (meth)acrylates.

Moreover, a copolymer based on polysulfonic acid, preferably acrylamidoalkylsulfonic acid and/or a salt thereof, and one or more comonomers selected from cyclic N-vinylcarboxamides and linear N-vinylcarboxamides, or a crosslinked acrylamidoalkylsulfonic acid copolymer; a crosslinked homopolymer of acrylamidoalkylsulfonic acid and/or a salt thereof; a copolymer of acrylamidoalkylsulfonic acid and/or a salt thereof, and a comonomer selected from (meth)acrylamide, (meth)alkylacrylamide, (meth)hydroxyethylacrylamide, polyalkyleneoxide alkyl-modified (meth)acrylate, hydroxyethyl (meth)acrylate, and cation-modified (meth)acrylates; and the like can also be used preferred.

Among them, a carboxyvinyl polymer, an acrylamidoalkylsulfonic acid/beheneth-25 crosslinked copolymer, an acrylamidoalkylsulfonic acid/vinylpyrrolidone crosslinked copolymer, and an acrylamidoalkylsulfonic acid/alkylacrylamide crosslinked copolymer are particularly preferable.

The crosslinking density of the crosslinked water-swellable polymer is 0.01 to 1 mol %, preferably 0.02 to 0.8 mol %, and most preferably 0.05 to 0.5 mol %. In the present invention, a polymer capable of infinitely swelling with water is not suitable.

Examples of the microgel include a microgel obtained by dissolving a hydrophilic compound having gelation ability in water or an aqueous component, leaving the solution to cool to form a gel, and crushing the formed gel.

The hydrophilic compound having gelation ability is not particularly limited, so long as it is a water-soluble compound having gelation ability and is used in the cosmetic and pharmaceutical fields. Specific examples include, but not limited to, hydrophilic proteins having gelation ability such as gelatin and collagen, and hydrophilic polysaccharides such as agar, curdlan, scleroglucan, schizophyllan, gellan gum, alginic acid, carrageenan, mannan, pectin, and hyaluronic acid. In particular, gelatin, agar, curdlan, gellan gum, alginic acid, and carrageenan can be used preferably because they are not easily affected by salts and ions, and are capable of forming a stable gel. One of or two or more of the hydrophilic compounds having gelation ability can be used.

The microgel according to the present invention can be produced by the method disclosed in Japanese Patent No. 4979095 B, for example. Specifically, after the hydrophilic compound having gelation ability is dissolved in water or an aqueous component, it is left to cool and solidified to form a gel. The compound can be dissolved in water or an aqueous component by mixing, heating, or the like. Gelation (solidification) may be carried out by stopping the heating after dissolution and leaving the solution to stand (to stand still) until the temperature is lower than the gelation temperature (solidification temperature).

Then, the formed gel is processed with a homogenizer, disper, mechanical stirrer, or the like to crush the formed gel and obtain the desired microgel. In the present invention, the average particle size of the microgel is preferably 0.1 to 1,000 μm, more preferably about 1 to 300 μm, and even more preferably about 10 to 200 μm.

The amount of the crosslinked water-swellable polymer having a crosslinking density of 0.01 to 1 mol % blended in the cosmetic of the present invention is 0.01 to 2% by mass, preferably 0.02 to 1.5% by mass, and more preferably 0.05 to 1% by mass. When the blending amount is less than 0.01% by mass, a sufficient thickening effect may not be achieved. When the blending amount exceeds 5% by mass, stickiness may occur.

The amount of the microgel obtained by crushing a gel, which is formed of a hydrophilic compound having gelation ability, blended in the cosmetic of the present invention is 0.1 to 5% by mass, preferably 0.15 to 4% by mass, and more preferably 0.2 to 3% by mass. When the amount is less than 0.1% by mass, a sufficient gelation ability may not be achieved. When the blending amount exceeds 5% by mass, a rough texture may occur.

[Oil Component]

The oil component that forms the oil phase of the cosmetic according to the present invention can be selected from oil components conventionally used in cosmetics and the like, and is not particularly limited. For example, the oil component may be one or two or more selected from hydrocarbon oils, higher fatty acids, higher alcohols, synthetic ester oils, silicone oils, liquid fats and oils, solid fats and oils, waxes, oil-soluble drugs, and the like.

Examples of hydrocarbon oils include, but not limited to, isododecane, isohexadecane, isoparaffin, liquid paraffin, ozocerite, squalane, pristane, paraffin, ceresin. squalene, vaseline, and microcrystalline wax.

Examples of higher fatty acids include, but not limited to, lauric acid, myristic acid, palmitic acid, stearic acid, behenic acid, oleic acid, undecylenic acid, tallic acid, isostearic acid, linoleic acid, linolenic acid, eicosapentaenoic acid (EPA), and docosahexaenoic acid (DHA).

Examples of higher alcohols include, but not limited to: linear alcohols (such as lauryl alcohol, cetyl alcohol, stearyl alcohol, behenyl alcohol, myristyl alcohol, oleyl alcohol, and cetostearyl alcohol); and branched-chain alcohols (such as monostearylglycerin ether (batyl alcohol)-2-decyltetradecinol, lanolin alcohol, cholesterol, phytosterol, hexyldodecanol, isostearyl alcohol, and octyldodecanol).

Examples of synthetic ester oils include, but not limited to, octyl octanoate, nonyl nonanoate, cetyl octanoate, isopropyl myristate, octyl dodecyl myristate, isopropyl palmitate, butyl stearate, hexyl laurate, myristyl myristate, decyl oleate, hexyl decyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylene glycol monoisostearate, neopentyl glycol dicaprate, tripropylene glycol pivalate, diisostearyl malate, glyceryl di-2-heptylundecanoate, glyceryl diisostearate, trimethylolpropane tri-2-ethyl hexanoate, trimethylolpropane triisostearate, pentaerythritol tetra-2-ethylhexanoate, glycerin tri-2-ethylhexanoate, glyceryl trioctanoate, glycerin triisopalmitate, trimethylolpropane triisostearate, cetyl 2-ethyl hexanoate-2-ethylhexyl palmitate, glycerin trimyristate, tri-2-heptyl undecanoic acid glyceride, castor oil fatty acid methyl ester, oleyl oleate, aceto glyceride, 2-heptylundecyl palmitate, diisobutyl adipate, 2-octyldodecyl N-lauroyl-L-glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate.

Examples of silicone oils include, but not limited to, chain polysiloxanes (e.g., dimethylpolysiloxane, methylphenyl polysiloxane, diphenyl polysiloxane, etc.), ring polysiloxanes (e.g., octamethylcyclotetrasiloxane, decamethyl cyclopenta siloxane, dodecamethyl cyclohexa siloxane, etc.), silicone resins forming a three-dimensional network structure, silicone rubbers, various modified polysiloxanes (amino-modified polysiloxane, polyether-modified polysiloxane, alkyl-modified polysiloxane, fluorine-modified polysiloxane, etc.), and acryl silicones.

Examples of liquid fats include, but not limited to, avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, and triglycerol.

Examples of solid fats include, but not limited to, cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, and hydrogenated caster oil.

Examples of waxes include, but not limited to, beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, and POE hydrogenated lanolin alcohol ether.

In addition to the oil components listed above, the oil phase of the oil-in-water emulsion cosmetic according to the present invention may contain oily components commonly used in cosmetics within the range of not adversely affecting the effect of the present invention.

[Surfactant]

In the present invention, various surfactants and/or emulsifiers can be used alone or in combination as an emulsifier.

A surfactant can be suitably selected from nonionic surfactants, cationic surfactants, anionic surfactants, and amphoteric surfactants, and it is preferable that the overall HLB becomes 7 or more. Here, the HLB is an index indicating a balance between hydrophilicity and lypophilicity (a hydrophilic-lypophilic balance). In the present invention, it is a value calculated by the following equation of Oda and Teramura, et al.

HLB=(Σinorganic value/Σorganic value)×10

The “overall HLB being 7 or more” means that, for example, when x % by mass of a surfactant having an HLB of “a” and (100−x) % by mass of a surfactant having an HLB of “b” are used in combination, a value of the overall HLB=a·x/100+b·(100−x)/100 is 7 or more.

In the following description, POE means polyoxyethylene, and POP means polyoxypropylene, respectively.

Examples of nonionic surfactants include, but not limited to: POE sorbitan fatty acid esters such as POE-sorbitan monostearate, POE-sorbitan monooleate, and POE-sorbitan tetraoleate; POE sorbit fatty acid esters such as POE-sorbit monooleate, POE-sorbit pentaoleate, and POE-sorbit monostearate; POE glycerin fatty acid esters such as POE-glycerin monostearate, POE-glycerin monoisostearate, and POE-glycerin triisostearate; POE fatty acid esters such as POE-monooleate, POE-distearate, POE-monodioleate, and ethylene glycol stearate; POE alkyl ethers such as POE-lauryl ether, POE-oleyl ether, POE-stearyl ether, POE-behenyl ether, POE2-octyldodecyl ether, and POE-cholestanol ether; POE alkylphenyl ethers such as POE-octylphenyl ether, POE-nonylphenyl ether, POE-nonylphenyl ether, and POE-dinonyl phenyl ether; pluaronics such as bluronic; POE•POP alkyl ethers such as POE•POP-cetyl ether, POE•POP2-decyltetradecyl ether, POE•POP-monobutyl ether, POE•POP hydrogenated lanolin, and POE•POP-glycerin ether; tetra POE.tetra POP ethylenediamine condensates such as tetronic; POE castor oil derivatives or hydrogenated castor oil derivatives such as POE castor oil, POE hydrogenated castor oil, POE hydrogenated castor oil monoisostearate, POE hydrogenated castor oil triisostearate, POE hydrogenated castor oil monopyroglutamic acid monoisostearic acid diester, and POE hydrogenated castor oil maleic acid; bees wax lanolin derivatives such as POE sorbit bees wax; glycerin fatty acid esters such as glycerin monostearate; polyglycerin fatty acid esters such as diglycerin diisostearate, decaglyceryl monostearate, decaglyceryl monoisostearate, decaglyceryl monooleate, decaglyceryl dioleate, and decaglyceryl triisostearate; sorbitan fatty acid esters such as sorbitan monooleate, sorbitan monoisostearate, sorbitan monostearate, sorbitan sesquioleate, sorbitan trioleate, diglycerol sorbitan penta-2-ethylhexylate, diglycerol sorbitan tetra-2-ethylhexylate; alkanolamides such as coconut oil fatty acid diethanolamide, lauric acid monoethanolamide, and fatty acid isopropanolamide; POE propylene glycol fatty acid ester, POE alkylamine, POE fatty acid amide, sucrose fatty acid ester, POE nonylphenylformaldehyde condensate, alkylethoxydimethylamine oxide, trioleyl phosphate; and dimethicone copolyols such as POE-modified dimethylpolysiloxane and POE•POP-modified dimethylpolysiloxane.

Examples of anionic surfactants include, but not limited to: higher fatty acid salts such as potassium stearate and potassium behenate; alkyl ether carboxylic acid salts such as sodium POE lauryl ether carboxylate; N-acyl-L-glutamic acid salts such as N-stearoyl-L-glutamic acid monosodium salt; higher alkyl sulfuric acid ester salts such as sodium lauryl sulfate and potassium lauryl sulfate; alkyl ether sulfuric acid ester salts such as POE triethanolamine lauryl sulfate and POE sodium lauryl sulfate; N-acyl sarcosinic acid salts such as sodium lauroyl sarcosinate; higher fatty acid amide sulfonates such as N-myristoyl-N-methyltaurine sodium; alkyl phosphoric acid salts such as sodium stearyl phosphate; alkyl ether phosphoric acid salts such as sodium POE oleyl ether phosphate and sodium POE stearyl ether phosphate; sulfosuccinic acid salts such as sodium di-2-ethylhexyl sulfosuccinate, sodium monolauroyl monoethanolamide polyoxyethylene sulfosuccinate, and sodium lauryl polypropylene glycol sulfosuccinate; alkylbenzenesulfonic acid salts such as sodium linear dodecylbenzenesulfonate, linear dodecylbenzene, triethanolamine sulfonate, and linear dodecylbenzenesulfonic acid; and higher fatty acid ester sulfuric acid ester salts such as hydrogenated coconut oil fatty acid glycerin sodium sulfate.

Examples of cationic surfactants include, but not limited to, alkyltrimethylammonium salts such as stearyltrimethylammonium chloride and lauryltrimethylammonium chloride, dialkyldimethylammonium salts such as distearyldimethylammonium chloride, poly(N,N-dimethyl-3,5-methylenepiperidinium) chloride, alkylpyridinium salts such as cetylpyridinium chloride, alkyl quaternary ammonium salts, alkyldimethylbenzylammonium salts, alkylisoquinolinium salts, dialkyl morphonium salts, POE alkylamines, alkylamine salts, polyamine fatty acid derivatives, amyl alcohol fatty acid derivatives, benzalkonium chloride, and benzethonium chloride.

Examples of amphoteric surfactants include, but not limited to: imidazoline type amphoteric surfactants such as 2-undecyl-N,N,N-(hydroxyethyl carboxymethyl)-2-imidazoline sodium salt and 2-cocoyl-2-imidazolinium hydroxide-1-carboxyethyloxy 2 sodium salt; and betaine type surfactants such as 2-heptadecyl-N-carboxymethyl-N-hydroxyethyl imidazolinium betaine, lauryldimethylaminoacetic acid betaine, alkyl betaine, amide betaine, and sulfobetaine.

The emulsifier is not particularly limited so long as it is commonly used in cosmetics, and, for example, a polymer that functions as a polymer emulsifier can be used. Examples of such polymers include acrylic acid/alkyl methacrylate copolymers, and commercially available products known as Carbopol 1342, Pemulen TR-1, and Pemulen TR-2 can be used.

[Water]

The cosmetic according to the present invention comprises water as the aqueous component. This water is not limited in particular. For example, purified water, ion-exchanged water, or tap water can be used.

Aqueous components commonly used in cosmetics other than water, such as water-soluble alcohol and the like, can be blended to the aqueous phase of the cosmetic according to the present invention within the range of not adversely affecting the effect of the present invention.

Examples of water-soluble alcohols include, but not limited to, lower alcohols, polyhydric alcohols, polyhydric alcohol polymers, dihydric alcohol alkyl ethers, dihydric alcohol ether esters, glycerin monoalkyl ethers, and sugar alcohols.

Examples of lower alcohols include, but not limited to, ethanol, propanol, isopropanol, isobutyl alcohol, and t-butyl alcohol.

Examples of polyhydric alcohols include, but not limited to: dihydric alcohols (e.g., dipropylene glycol, 1,3-butylene glycol, ethylene glycol, trimethylene glycol, 1,2-butylene glycol, tetramethylene glycol, 2,3-butylene glycol, pentamethylene glycol, 2-butene-1,4-diol, hexylene glycol, and octylene glycol); trihydric alcohols (e.g., glycerin and trimethylolpropane); tetrahydric alcohols (e.g., diglycerin and pentaerythritol such as 1,2,6-hexanetriol); pentahydric alcohols (e.g., xylitol and triglycerin); hexahydric alcohols (e.g., sorbitol and mannitol); polyhydric alcohol polymers (e.g., diethylene glycol, dipropylene glycol-triethylene glycol, polypropylene glycol, tetraethylene glycol, diglycerin-triglycerin, tetraglycerin, and polyglycerin); dihydric alcohol alkylethers (e.g., ethylene glycol monomethyl ether, ethylene glycol monoethyl ether, ethylene glycol monobutyl ether, ethylene glycol monophenyl ether, ethylene glycol monohexyl ether, ethylene glycol mono 2-methyl hexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether, diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, and dipropylene glycol butyl ether); dihydric alcohol ether esters (e.g., ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, and propylene glycol monophenyl ether acetate); glycerin mono alkyl ethers (e.g., xylyl alcohol, selachyl alcohol, and batyl alcohol); sugar alcohols (e.g., maltotriose, mannitol, sucrose, erythritol, glucose, fructose, starch amylolysis sugar, maltose, and alcohol prepared by the reduction of starch amylolysis sugar); glysolid; tetrahydro furfuryl alcohol; POE-tetrahydro furfuryl alcohol; POP-butyl ether; POP/POE-butyl ether; tripolyoxypropylene glycerin ether; POP-glycerin ether; POP-glycerin ether phosphoric acid; POP/POE-pentane erythritol ether; and polyglycerin.

Other components commonly used in cosmetics can be blended to the cosmetic of the present invention within the range of not adversely affecting the effect of the present invention. Examples of such components include, but not limited to, moisturizers, ultraviolet absorbents, medicinal components, transdermal absorption promoters, sequestrants, powder components, vitamins, pH adjusters, antioxidants, antiseptic agents, antibacterial agents, neutralizers, perfumes and pigments.

The cosmetic of the present invention can be in a product form such as lotions, emulsions, beauty serums, creams, and makeup bases. Lotions, emulsions, and beauty serums are particularly preferred.

The cosmetic according to the present invention can be prepared by an ordinary method. For example, it may be prepared by: mixing and dissolving oil phase components; and adding the solution while stirring to aqueous phase components to emulsify.

### EXAMPLES

The present invention will now be explained in more detail with Examples, but the scope of the present invention is not limited by these Examples. Moreover, the blending amounts in the Examples is in “% by mass” unless specified otherwise.

First, the methods for synthesizing precisely synthesized sodium polyacrylates used in the Examples are described.

### Synthesis Example 1

Acrylic acid (2511 mg) and V-501 (0.17 mg) were dissolved in ion-exchanged water (9 mL), a methanol solution (1 ml) in which CPD (0.17 mg) had been dissolved was added, and a polymerization reaction was carried out under an argon atmosphere at 60° C. for 24 hours. After the polymerization reaction, sodium hydroxide aqueous solution was added for adjusting the pH to 6.0 to 7.0, and the mixture was dialyzed against purified water for 4 days. After freeze-drying, precisely synthesized sodium polyacrylate-1 (1.82 g, yield: 72%) was collected. As a result of GPC analysis, the weight-average molecular weight was 7,300,000, and the molecular weight distribution was 1.2.

### Synthesis Example 2

Acrylic acid (2514 mg), methylenebisacrylamide (9.6 μg), and V-501 (0.17 mg) were dissolved in ion-exchanged water (9 ml), a methanol solution (1 ml) in which CPD (0.17 mg) had been dissolved was added, and a polymerization reaction was carried out under an argon atmosphere at 60° C. for 24 hours. After the polymerization reaction, sodium hydroxide aqueous solution was added for adjusting the pH to 6.0 to 7.0, and the mixture was dialyzed against purified water for 4 days. After freeze-drying, precisely synthesized sodium polyacrylate-2 (1.99 g, yield: 79%) was collected. As a result of GPC analysis, the weight-average molecular weight was 3,260,000, and the molecular weight distribution was 1.7.

### Synthesis Example 3

Acrylic acid (120 g) and V-501 (0.12 g) were dissolved in ion-exchanged water (760 g), a methanol solution (95 g) in which CPD (0.12 g) had been dissolved was added, and a polymerization reaction was carried out under an argon atmosphere at 60° C. for 96 hours. After the polymerization reaction, sodium hydroxide aqueous solution was added for adjusting the pH to 6.0 to 7.0, and the mixture was purified by re-precipitation using water/acetone. After drying under reduced pressure, precisely synthesized sodium polyacrylate-3 (75.6 g, yield: 63%) was collected. As a result of GPC analysis, the weight-average molecular weight was 695,000, and the molecular weight distribution was 1.3.

Other components marked with * in Tables 1 and 2 below are as follows.

*1: Pemulen TR-2 (manufactured by BF Goodrich)
*2, 3: Sodium salt of polyacrylic acid (partially neutralized)

The content of molecular species having a molecular weight of 10,000,000 or more and the content of the compound having a molecular weight equal three times or greater than the weight-average molecular weight are both more than 10% by mass (already analyzed in Patent Literature 2).

### Test Example 1

Oil-in-water emulsion cosmetics (beauty serums) having the formulations shown in Tables 1 and 2 were prepared by the following production methods, and the physical properties were evaluated by the following methods. Furthermore, the actual application tests were carried out by professional panelists for the following items (1) to (6). The results are shown in Tables 1 and 2.

<Production Method>

Dimethicone and triethylhexanoin were mixed and dissolved (=mixture A), and the remaining components other than potassium hydroxide were uniformly dissolved (=mixture B). The mixture A was gradually added to the mixture B, and the resultant was mixed by a homogenizer. Furthermore, potassium hydroxide was added and processed by the homogenizer, and thus a predetermined beauty serum was obtained.

<Physical Property Evaluation>

Viscosity

Each composition was stored at 25° C. and then rotated (12 rpm) for one minute to measure the viscosity value (mPa·s) with a B-type rotational viscometer (Vismetron viscometer, manufactured by Shibaura Systems Co., Ltd.).

pH

The pH at 25° C. was measured with a pH meter (HORIBA pH METER F-52, manufactured by HORIBA Ltd.).

Gradient of First Normal Stress Difference

The first normal stress difference of each composition was calculated, and then the calculated first normal stress difference was divided by the shear velocity (see Patent Literature 1). The value obtained thereby was calculated as the gradient of the first normal stress difference. The measurement value of the first normal stress difference (Pa) with respect to the shear velocity (s⁻¹) at the shear velocity of 100 s⁻¹ or more was plotted, and the slope by linear approximation was calculated as the slope of the first normal stress difference (Pa·s).

Viscoelastic ratio (tan δ)

The change in elastic modulus of the composition corresponding to the change in strain upon applying a frequency of 1 Hz was measured, and a loss elastic modulus G″ and a storage elastic modulus G′ under specific strain conditions were calculated. The value obtained as the ratio therebetween, i.e., G″/G′, was regarded as the viscoelastic ratio.

<Actual Application Test>

Ten professional panelists applied the test compositions to their faces, and evaluated whether the test compositions were effective or not in: (1) rich feeling, (2) full-bodied feeling, (3) freshness, (4) non-stringiness, (5) good spreadability, and (6) non-stickiness. The answers were totalized according to the following criteria, and the results are shown in the tables.

A: Nine or more panelists answered “effective”.

B: Seven or more and eight or fewer panelists answered “effective”.

C: Five or more and six or fewer panelists answered “effective”.

D: Four or fewer panelists answered “effective”.

In the present invention, A and B were determined as being acceptable, and C and D were determined as being unacceptable.

TABLE 1

| | | | Comparative | | Comparative | | Comparative | |
|---|---|---|---|---|---|---|---|---|
| | | | Example 1 | Example 1 | Example 2 | Example 2 | Example 3 | Example 3 |
| Formulation | (b) | Carboxyvinyl polymer | 0.1 | 0.1 | — | — | — | — |
| | | Crosslinked sodium N,N- | — | — | 0. [symbol] | 0. [symbol] | — | — |
| | | dimethylacrtlamide-2-acrylamide- | | | | | | |
| | | 2-menthylpropanesulfonic acid copolymer | | | | | | |
| | | (Acryloyldimethyltaurate/VP) copolymer | — | — | — | — | 0.4 | 0. [symbol] |
| | | (Acryloyldimethyltaurine salt/beheneth-2 [symbol] | — | — | — | — | — | — |
| | | methacrylate) copolymer | | | | | | |
| | | (Acrylates/Alkyl (C10-30) acrylate | 0.07 | 0.07 | 0.07 | 0.07 | 0.07 | 0.07 |
| | | crosspolymer * [symbol] | | | | | | |
| | | A [symbol] er | — | — | — | — | — | — |
| | | Precisely synthesized sodium polyacrylate | — | 0. [symbol] | — | 0. [symbol] | — | 0. [symbol] |
| | | (Synthesis Example 1) | | | | | | |
| | Other | Glycerin | [symbol] . [symbol] | [symbol] . [symbol] | [symbol] . [symbol] | [symbol] . [symbol] | [symbol] . [symbol] | [symbol] . [symbol] |
| | components | 1, [symbol] -Butylene glycol | [symbol] . [symbol] | [symbol] . [symbol] | [symbol] . [symbol] | [symbol] . [symbol] | [symbol] . [symbol] | [symbol] . [symbol] |
| | | Dimethicone | [symbol] . [symbol] | [symbol] . [symbol] | [symbol] . [symbol] | [symbol] . [symbol] | [symbol] . [symbol] | [symbol] . [symbol] |
| | | Triethylhexanoin | 1.0 | 1.0 | 1.0 | 1.0 | 1.0 | 1.0 |
| | | Potassium hydroxide | 0. [symbol] | 0. [symbol] | 0. [symbol] | 0. [symbol] | 0. [symbol] | 0. [symbol] |
| | | Methylparaben | Suitable | Suitable | Suitable | Suitable | Suitable | Suitable |
| | | | amount | amount | amount | amount | amount | amount |
| | | EDTA-2Na | Suitable | Suitable | Suitable | Suitable | Suitable | Suitable |
| | | | amount | amount | amount | amount | amount | amount |
| | | Ion-e [symbol] hanged water | Balance | Balance | Balance | Balance | Balance | Balance |

| | | | | | | | |
|---|---|---|---|---|---|---|---|
| | Total | 100.0 | 100.0 | 100.0 | 100.0 | 100.0 | 100.0 |
| Evaluation | Viscosity(Pa · s) | 0. [symbol] | 0. [symbol] | 1.01 | 0. [symbol] | [symbol] . [symbol] | 1. [symbol] |
| | pH | 6.8 | [symbol] . [symbol] | [symbol] .2 | [symbol] .7 | 6. [symbol] | 8.9 |
| | Gradient of the first normal stress difference | 0.07 [symbol] | 0.27 [symbol] | 0. [symbol] 2 | 0. [symbol] | 0.10 [symbol] | 0.2 [symbol] |
| | Viscoelastic ratio | 0. [symbol] 1 | [symbol] . [symbol] | [symbol] . [symbol] 1 | 1.2 [symbol] | [symbol] .1 [symbol] | 1.0 [symbol] |
| | (1) Rich feeling | C | A | C | A | C | A |
| | (2) Full-bodied feeling | C | A | C | A | C | A |
| | (3) Freshness | B | B | A | B | B | B |
| | (4) Non-stringiness | A | A | A | A | A | A |
| | (5) Spreadability | B | A | B | A | B | B |
| | (6) Non-stickiness | B | A | B | A | B | A |

| | | | Comparative | | Comparative | |
|---|---|---|---|---|---|---|
| | | | Example 4 | Example 4 | Example [symbol] | Example 5 |
| Formulation | (b) | Carboxyvinyl polymer | — | — | — | — |
| | | Crosslinked sodium N,N- | — | — | — | — |
| | | dimethylacrtlamide-2-acrylamide- | | | | |
| | | 2-menthylpropanesulfonic acid copolymer | | | | |
| | | (Acryloyldimethyltaurate/VP) copolymer | — | — | — | — |
| | | (Acryloyldimethyltaurine salt/beheneth-2 [symbol] | 0. [symbol] | 0. [symbol] | — | — |
| | | methacrylate) copolymer | | | | |
| | | (Acrylates/Alkyl (C10-30) acrylate | 0.07 | 0.07 | 0.07 | 0.07 |
| | | crosspolymer * [symbol] | | | | |
| | | A [symbol] er | — | — | 1.0 | 1.0 |
| | (a) | Precisely synthesized sodium polyacrylate | — | 0. [symbol] | — | 0. [symbol] |
| | | (Synthesis Example 1) | | | | |
| | Other | Glycerin | [symbol] . [symbol] | [symbol] . [symbol] | [symbol] . [symbol] | [symbol] . [symbol] |
| | components | 1, [symbol] -Butylene glycol | [symbol] . [symbol] | [symbol] . [symbol] | [symbol] . [symbol] | [symbol] . [symbol] |
| | | Dimethicone | [symbol] . [symbol] | [symbol] . [symbol] | [symbol] . [symbol] | [symbol] . [symbol] |
| | | Triethylhexanoin | 1.0 | 1.0 | 1.0 | 1.0 |
| | | Potassium hydroxide | 0. [symbol] | 0. [symbol] | 0. [symbol] | 0. [symbol] |
| | | Methylparaben | Suitable | Suitable | Suitable | Suitable |
| | | | amount | amount | amount | amount |
| | | EDTA-2Na | Suitable | Suitable | Suitable | Suitable |
| | | | amount | amount | amount | amount |
| | | Ion-e [symbol] hanged water | Balance | Balance | Balance | Balance |

| | | | | | |
|---|---|---|---|---|---|
| | Total | 100.0 | 100.0 | 100.0 | 100.0 |
| Evaluation | Viscosity(Pa · s) | 1. [symbol] | [symbol] . [symbol] | 2.31 | [symbol] . [symbol] |
| | pH | 0. [symbol] | [symbol] .0 | 8. [symbol] | [symbol] .7 |
| | Gradient of the first normal stress difference | 0.11 [symbol] | 0. [symbol] 6 | 0.0 [symbol] | 0. [symbol] |
| | Viscoelastic ratio | 1. [symbol] | [symbol] | 0. [symbol] | 0. [symbol] |
| | (1) Rich feeling | C | A | C | A |
| | (2) Full-bodied feeling | C | A | C | A |
| | (3) Freshness | B | B | A | B |
| | (4) Non-stringiness | A | A | A | A |
| | (5) Spreadability | B | B | B | A |
| | (6) Non-stickiness | B | A | B | A |

[symbol]

indicates data missing or illegible when filed

As shown in Table 1, the beauty serum containing (b): (acrylates/alkyl (C10-30) acrylate) crosspolymer and a carboxyvinyl polymer, which are not precisely synthesized polymers, as thickeners were excellent in freshness, non-stringiness, spreadability, and non-stickiness, but not sufficient in rich feeling and full-bodied feeling (Comparative Example 1). On the other hand, in the beauty serum in which the precisely synthesized sodium polyacrylate (Synthesis Example 1) was added to the formulation of Comparative Example 1, the viscoelastic ratio (tan δ) and the gradient of the first normal stress difference significantly increased, and thus extremely good rich feeling and full-bodied feeling could be achieved in addition to the effects such as freshness and the like (Example 1).

Similarly, in any of the beauty serums containing (b): (acrylates/alkyl (C10-30) acrylate) crosspolymer in combination with a crosslinked sodium N,N-dimethylacrylamide-2-acrylamido-2-methylpropanesulfonic acid copolymer (Comparative Example 2), (acryloyldimethyltaurate/VP) copolymer (Comparative Example 3), (acryloyldimethyltaurine salt/beheneth-25 methacrylate) copolymer (Comparative Example 4), or agar (Comparative Example 5), which are not precisely synthesized polymers, as thickeners, neither rich feeling nor full-bodied feeling was achieved. However, in all beauty serums in which (a): precisely synthesized sodium polyacrylate (Synthesis Example 1) was added to these formulations, the viscoelastic ratio and the gradient of the first normal stress difference significantly increased, and thus extremely excellent rich feeling and full-bodied feeling were exhibited (Examples 2 to 5).

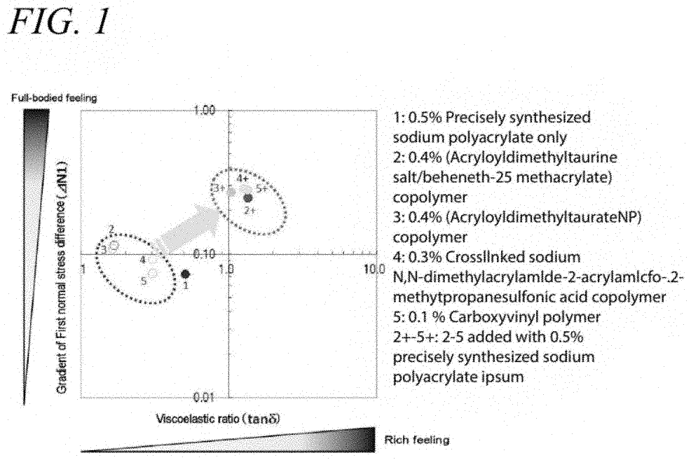

FIG. 1 shows the viscoelastic ratio and the gradient of the first normal stress difference when the component (a) and the component (b) are used alone or in combination. Generally, the beauty serums plotted in the first quadrant of FIG. 1 (the viscoelastic ratio is greater than 1.0, and the gradient of the first normal stress difference is greater than 0.10) have sufficient viscoelastic ratio and gradient of the first normal stress difference, and can be determined as being capable of exhibiting rich feeling and full-bodied feeling.

As shown in FIG. 1, the component (a) and the component (b) are plotted in the second quadrant or the third quadrant when used alone. However, when the component (a) and the component (b) are used in combination, the values on both the x-axis and the Y-axis becomes higher than the sum of the values obtained when the component (a) and the component (b) are used alone, and are plotted in the first quadrant.

Therefore, it was demonstrated that when the component (a) and the component (b) are used in combination, a synergistic effect between them is generated, and thus the viscoelastic ratio and the gradient of the first normal stress difference increase.

From the above results, it is revealed that when the precisely synthesized polymer of the present invention is added to various thickeners that are not molecularly controlled and are commonly used in cosmetics, the viscoelastic ratio and the gradient of the first normal stress difference of the system greatly increase by the synergistic effect of both components, and thus extremely excellent rich feeling and full-bodied feeling can be achieved while retaining freshness, good spreadability, non-stickiness, and non-stringiness.

### Test Example 2

Next, the polymer to be added was investigated.

Specifically, not only (a): the precisely synthesized sodium polyacrylate, but also sodium polyacrylate synthesized by an ordinary method was blended, and the effect thereof was compared and examined. The results are shown in Table 2.

TABLE 2

| | | | Compar- | | Compar- | Compar- | Compar- | Compar- |
|---|---|---|---|---|---|---|---|---|
| | | | ative | | ative | ative | ative | ative |
| | | | Example 4 | Example 4 | example [symbol] | Example 7 | example [symbol] | example [symbol] |
| Formulation | (b) | Carboxyvinyl polymer | — | — | — | — | — | — |
| | | Crosslinked sodium N,N-dimethylacrtlamide-2- | — | — | — | — | — | — |
| | | acrylamide-2-menthylpropanesulfonic | | | | | | |
| | | acid copolymer | | | | | | |
| | | (Acryloyldimethyltaurate/VP) copolymer | — | — | — | — | — | — |
| | | (Acryloyldimethyltaurine salt/beheneth-2 [symbol] | 0. [symbol] | 0. [symbol] | 0. [symbol] | 0. [symbol] | 0. [symbol] | 0. [symbol] |
| | | methacrylate) copolymer | | | | | | |
| | | (Acrylates/Alkyl (C10-30) acrylate | 0.07 | 0.07 | 0.07 | 0.07 | 0.07 | 0.07 |
| | | crosspolymer * [symbol] | | | | | | |
| | | A [symbol] er | — | — | — | — | — | — |
| | (a) | Precisely synthesized sodium | — | 0.5 | — | — | — | — |
| | | polyacrylate (Synthesis Example 1) | | | | | | |
| | | Ordinarily synthesized sodium | — | — | 0. [symbol] | 0. [symbol] | — | — |
| | | polyacrylate-1 *2 | | | | | | |
| | | Ordinarily synthesized sodium | — | — | — | — | 0. [symbol] | 0.5 |
| | | polyacrylate-2 *3 | | | | | | |
| | Other | Glycerin | [symbol] .0 | [symbol] .0 | [symbol] .0 | [symbol] .0 | [symbol] .0 | [symbol] .0 |
| | components | 1, [symbol] -Butylene glycol | 3.0 | 3.0 | 3.0 | 3.0 | 3.0 | 3.0 |
| | | Dimethicone | 3.0 | 3.0 | 3.0 | 3.0 | 3.0 | 3.0 |
| | | Triethylhexanoin | 1.0 | 1.0 | 1.0 | 1.0 | 1.0 | 1.0 |
| | | Potassium hydroxide | 0. [symbol] 0 | 0. [symbol] 0 | 0. [symbol] 0 | 0. [symbol] 0 | 0. [symbol] 0 | 0. [symbol] 0 |
| | | Methylparaben | Suitable | Suitable | Suitable | Suitable | Suitable | Suitable |
| | | | amount | amount | amount | amount | amount | amount |
| | | EDTA-2Na | Suitable | Suitable | Suitable | Suitable | Suitable | Suitable |
| | | | amount | amount | amount | amount | amount | amount |
| | | Ion-e [symbol] hanged water | Balance | Balance | Balance | Balance | Balance | Balance |

| | | | | | | | |
|---|---|---|---|---|---|---|---|
| | Total | 100.0 | 100.0 | 100.0 | 100.0 | 100.0 | 100.0 |
| Evaluation | Viscosity(Pa · s) | 1.55 | 1.04 | 0. [symbol] | 1.25 | 2.50 | 3.52 |
| | pH | 6.4 | 7.0 | 7.2 | 7.5 | 6.3 | 6.2 |
| | Gradient of the first normal stress difference | 0.115 | 0.24 [symbol] | 0.472 | 1.5 [symbol] | 0.4 [symbol] 1 | 0.898 |
| | Viscoelastic ratio | 0.17 | 1. [symbol] | 0. [symbol] 0 | 0.40 | 0. [symbol] 9 | 0. [symbol] 0 |
| | (1) Rich feeling | C | A | B | A | C | B |
| | (2) Full-bodied feeling | C | A | B | A | C | C |
| | (3) Freshness | B | B | D | D | B | C |
| | (4) Non-stringiness | A | A | D | D | C | D |
| | (5) Spreadability | B | B | A | A | B | B |
| | (6) Non-stickiness | B | A | B | C | B | C |

[symbol]

indicates data missing or illegible when filed

In the bases to which ordinarily synthesized sodium polyacrylate-1 were added, the gradient of the first normal stress difference extremely increased in addition to the viscoelastic ratio, and excellent rich feeling and full-bodied feeling were achieved; however, stringiness was caused, and freshness was impaired (Comparative Examples 6 and 7). Moreover, in the bases to which ordinarily synthesized sodium polyacrylate-2 was added, the gradient of the first normal stress difference extremely increased in addition to the viscoelastic ratio, stringiness was caused, and rich feeling, full-bodied feeling, and freshness tended to be impaired (Comparative Examples 8 and 9).

Therefore, it was demonstrated that the effect achieved by adding the precisely synthesized sodium polyacrylate is due to the point that the polymer is molecularly controlled; that is, the weight-average molecular weight being 500,000 to 8,000,000, the content of compounds having a molecular weight of 10,000,000 or more being 10% by mass or less, and the polymer being linear.

### Test Example 3

Next, the effect of the precisely synthesized polymer according to the present invention with respect to an emulsion was examined.

Oil-in-water emulsion cosmetics (emulsions) having the formulations shown in Table 3 were prepared by an ordinary method, and the physical properties and the feeling upon use were evaluated in the same manner as in Test Example 1. The results are shown in Table 3.

TABLE 3

| | | | Compar- | | | Compar- | | | Compar- | | |
|---|---|---|---|---|---|---|---|---|---|---|---|
| | | | ative | | | ative | | | ative | | |
| | | | Exam- | Exam- | Exam- | Exam- | Exam- | Exam- | Exam- | Exam- | Exam- |
| | | | ple 10 | ple 6 | ple 7 | ple 11 | ple 8 | ple 9 | ple 12 | ple 10 | ple 11 |
| Formu- | (b) | Carboxyvinyl polymer | 0.1 [symbol] | 0.1 [symbol] | 0.1 [symbol] | 0.1 [symbol] | 0.1 [symbol] | 0.1 [symbol] | — | — | — |
| lation | (a) | Precisely synthesized | — | 0. [symbol] | 0. [symbol] | — | — | — | — | — | — |
| | | sodium polyacrylate | | | | | | | | | |
| | | (Synthesis Example 1) | | | | | | | | | |
| | | Precisely synthesized | — | — | — | — | 0. [symbol] | 0. [symbol] | — | — | — |
| | | sodium polyacrylate | | | | | | | | | |
| | | (Synthesis Example 2) | | | | | | | | | |
| | | Precisely synthesized | — | — | — | — | — | — | — | 0. [symbol] | 0. [symbol] |
| | | sodium polyacrylate | | | | | | | | | |
| | | (Synthesis Example 3) | | | | | | | | | |
| | Other | Glycerin | 4 | 4 | 4 | 4 | [symbol] | [symbol] | 5 | [symbol] | [symbol] |
| | components | Dipropylene glycol | 7 | 7 | 7 | 7 | 7 | 7 | 5 | 5 | 5 |
| | | Butylene glycol | [symbol] | [symbol] | [symbol] | [symbol] | [symbol] | [symbol] | — | — | — |
| | | Alcohol | — | — | — | — | — | — | 5 | [symbol] | [symbol] |
| | | Xanthan gum | 0.0 [symbol] | 0.0 [symbol] | 0.0 [symbol] | 0.0 [symbol] | 0.0 [symbol] | 0.0 [symbol] | 0.07 | 0.07 | 0.07 |
| | | Hydrogenated polydecene | [symbol] | [symbol] | [symbol] | [symbol] | [symbol] | 4 | 3 | 3 | 3 |
| | | Phy [symbol] teryl m [symbol] damiate | — | — | — | — | — | — | 0. [symbol] | 0. [symbol] | 0. [symbol] |
| | | Pentaerythrityl | — | — | — | — | — | — | [symbol] | [symbol] | [symbol] |
| | | tetraethylhexanoate | | | | | | | | | |
| | | Cetyl ethylhexanoate | [symbol] | [symbol] | [symbol] | [symbol] | [symbol] | [symbol] | — | — | — |
| | | Vaseline | — | — | — | — | — | — | 0.5 | 0.5 | 0. [symbol] |
| | | Hydrogenated [symbol] oil | 1 | 1 | 1 | 1 | 1 | 1 | — | — | — |
| | | Dimethicone | 2.7 | 2.7 | 2.7 | 2.7 | 2.7 | 2.7 | 1 | 1 | 1 |
| | | Behenyl alcohol | 1 | 1 | 1 | 1 | 1 | 1 | 1.4 | 1.4 | 1.4 |
| | | Batyl alcohol | 0. [symbol] | 0. [symbol] | 0. [symbol] | 0. [symbol] | 0. [symbol] | 0. [symbol] | 0.52 | 0.52 | 0.52 |
| | | Isostearic acid | 0.4 | 0.4 | 0.4 | 0.4 | 0.4 | 0.4 | — | — | — |
| | | Stearic acid | 0. [symbol] | 0. [symbol] | 0. [symbol] | 0. [symbol] | 0. [symbol] | 0. [symbol] | — | — | — |
| | | Behenic acid | 0. [symbol] | 0. [symbol] | 0. [symbol] | 0. [symbol] | 0. [symbol] | 0. [symbol] | — | — | — |
| | | Glyceryl stearates | 0.5 | 0. [symbol] | 0. [symbol] | 0. [symbol] | 0. [symbol] | 0. [symbol] | — | — | — |
| | | PEG- [symbol] glyceryl stearate | 1. [symbol] | 1. [symbol] | 1. [symbol] | 1. [symbol] | 1. [symbol] | 1. [symbol] | — | — | — |
| | | Beherieth-20 | — | — | — | — | — | — | 0.82 | 0.82 | 0. [symbol] 2 |
| | | Citric acid | — | — | — | — | — | — | 0.02 | 0.02 | 0.02 |
| | | Sodium Citrate | — | — | — | — | — | — | 0.0 [symbol] | 0.0 [symbol] | 0.0 [symbol] |
| | | Triethanolamine | — | — | — | 1. [symbol] | 1. [symbol] | 1. [symbol] | — | — | — |
| | | Phenoylethanol | 0. [symbol] | 0. [symbol] | 0. [symbol] | 0. [symbol] | 0. [symbol] | 0. [symbol] | 0. [symbol] | 0. [symbol] | 0. [symbol] |
| | | Potassium hydroxide | 0.12 | 0.12 | 0.12 | — | — | — | — | — | — |
| | | Ion-e [symbol] hange water | Balance | Balance | Balance | Balance | Balance | Balance | Balance | Balance | Balance |

| | | | | | | | | | | |
|---|---|---|---|---|---|---|---|---|---|---|
| | Total | 100.0 | 100.0 | 100.0 | 100.0 | 100.0 | 100.0 | 100.0 | 100.0 | 100.0 |
| Evalu- | Gradient of the first | 0.217 | 0.20 [symbol] | 0. [symbol] 43 | 0.1 [symbol] | 0.385 | 0. [symbol] 12 | 0.01 [symbol] | 0.17 [symbol] | 0.2 [symbol] 7 |
| ation | normal stress difference | | | | | | | | | |
| | Viscoelastic ratio | 0. [symbol] 0 | 0.41 | 0. [symbol] 2 | 0.41 | 0. [symbol] | 0.7 [symbol] | 0.27 | 0.31 | 0. [symbol] |
| | (1) Rich feeling | B | A | A | B | A | A | B | A | A |
| | (2) Full-bodied feeling | B | A | A | B | A | A | B | A | A |
| | (3) Freshness | C | B | B | C | B | B | B | B | B |
| | (4) Non-stringiness | A | A | A | A | A | A | A | A | A |
| | (5) Spreadability | C | A | A | C | A | A | C | A | A |
| | (6) Non-stickness | B | B | B | B | B | B | B | B | B |

[symbol]

indicates data missing or illegible when filed

As shown in Table 3, the emulsions solely blended with (b): carboxyvinyl polymer, which is not a precisely synthesized polymer, as a thickener was excellent in rich feeling, full-bodied feeling, non-stringiness, and non-stickiness, but inferior in freshness and insufficient in spreadability (Comparative Examples 10 to 12). On the other hand, in the emulsions in which the precisely synthesized sodium polyacrylate was added to the formulations of these Comparative Examples, the viscoelastic ratio (tan δ) and the gradient of the first normal stress difference significantly increased in accordance with the amount of sodium polyacrylate added, resulting in even better rich feeling and full-bodied feeling (Examples 6 to 11). Furthermore, in these Examples, freshness and spreadability were also significantly improved (Examples 6 or 7 with respect to Comparative Example 10, Example 8 or 9 with respect to Comparative Example 11, and Example 10 or 11 with respect to Comparative Example 12).

Therefore, it was demonstrated that when the component (a) according to the present invention and the component (b) are used in combination in emulsions, the viscoelastic ratio and the gradient of the first normal stress difference increase; thus, rich feeling and full-bodied feeling are enhanced and, freshness and good spreadability are imparted, too.

### Test Example 4

Furthermore, sodium polyacrylate was produced by a polymerization method different from the RAFT polymerization method used in the present application, and the thread length was measured. As for this method, the polymerization method that uses 2-mercaptoethanol as a chain transfer agent as disclosed in Example 1 of Japanese Patent No. 5076428 B was employed.

<Method for Producing Sodium Polyacrylate>

An aqueous solution of a monomer mixture containing 69 g (0.94 mol) of 98% acrylic acid, 245.5 g (0.94 mol) of a 36% aqueous solution of sodium acrylate, and 205 g of pure water was added to a hermetically closed three-necked flask and, while stirring the aqueous solution, dissolved oxygen was removed by argon. Under argon purge, 0.14 g of 2,2′-azobis(2-methylpropionamidine) dihydrochloride and 0.00185 g of 2-mercaptoethanol were respectively diluted with argon-purged pure water and added as 1% aqueous solutions to the monomer mixture with a syringe, and thus an aqueous solution was prepared.

Next, the monomer mixture in which the dissolved oxygen is sufficiently replaced with argon was added to an 85.7 φ polystyrene dish, lidded, and thermally polymerized in a thermostat (Model ADP300 manufactured by Yamato Scientific Co., Ltd.) at 60° C., and obtained a gel-like substance as in Example 1 of Japanese Patent No. 5076428 B (in Example 1 of Japanese Patent No. 5076428 B, the temperature was raised to about 60° C. by UV lamp irradiation, but the same conditions were reproduced with a thermostat).

In order to remove the residual monomers, the gel-like substance was stirred and dissolved in pure water, and then dialyzed with a dialysis tube (Fisherbrand regenerated cellulose having a pore size of 10 Å). After dialysis, white polymer powder was collected by freeze-drying (FDU2100, Tokyo Rikakikai Co., Ltd.).

<Physical Property Evaluation>

Stringiness

The apparatus and conditions described in Patent Literature 2 were used for evaluations. Specifically, a 1% by mass aqueous solution of the obtained sodium polyacrylate was prepared and stored in a container at room temperature. The container was set on a texture analyzer (TA.XT plus, manufactured by Stable Micro Systems), a round disk having a diameter of about 1 cm was uniformly and lightly brought into contact with the surface of the aqueous solution, and then the container was lowered at a velocity of 5 mm/sec to observe how the solution forms a thread. The distance that the container was lowered until the thread of the solution was cut was measured as a “thread length.” This thread length is a value that serves an index of stringiness of the polymer compound, and a greater value indicates a greater stringiness. When the thread length was 10 mm or less, the stringiness was determined as being low.

Aqueous Solution Viscosity Measurement

39.92 g of pure water was added to a 50 ml glass screw tube. 0.08 g of sodium polyacrylate was added thereto, and the mixture was stirred for 10 minutes with a planetary mixer (Sinky Corporation, ARE-100) to give a 0.2% by mass aqueous solution. The viscosity of the prepared solution was measured with a B-type viscometer under the condition of 20° C. and 30 rpm.

As a control, with respect to the precisely synthesized sodium polyacrylate-1 synthesized by the method disclosed in Synthesis Example 1 of the present description, 39.92 g of pure water was put into a 50 ml glass screw tube, 0.08 g of sodium polyacrylate was added thereto, and the mixture was stirred for 10 minutes with a planetary mixer (Sinky Corporation, ARE-100) to give a 0.2% by weight aqueous solution. After repeating the preparation for two times, the samples were put together in a 100 mL glass screw tube, and the viscosity was measured with a B-type viscometer under the condition of 20° C. and 30 rpm.

<Results>

With respect to the 1% aqueous solution of sodium polyacrylate produced by the polymerization method that was not the RAFT polymerization method, stringiness was clearly recognized even by visual observation (the viscous and thread-forming state were observed during the measurement), and the measured thread length was 12 mm. Moreover, the viscosity of the 0.2% by mass aqueous solution of the sodium polyacrylate was 560 mPa·s.

On the other hand, the precisely synthesized sodium polyacrylate-1 synthesized by the method of Synthesis Example 1 of the present application did not show any stringiness by visual observation, and the thread length was 6 mm. In addition, the viscosity of the 0.2% by mass aqueous solution of the sodium polyacrylate was 64.4 mPa·s. It is generally known that the stringiness of a polymer tends to correlate with viscosity and viscoelasticity.

Therefore, it was verified that the stringiness of a polymer greatly varies according to the synthesis method of the polymer, and when the RAFT polymerization method is used, a polymer having a much lower stringiness (than when a polymerization method that uses a chain transfer agent other than the RAFT agent is used) can be obtained.

Formulation examples of the cosmetics according to the present invention are listed in the following, but the present invention is not limited thereto. The following cosmetics were produced by ordinary methods unless specified otherwise.

### Formulation Example 1: Cosmetic Lotion

<Formulation>

| | Blending amount |
|---|---|
| Component | (% by mass) |
| Ethyl alcohol | 5 |
| Glycerin | 1 |
| 1,3-Butylene glycol | 5 |
| Polyoxyethylene polyoxypropylene | 0.2 |
| decyltetradecyl ether | |
| Sodium hexametaphosphate | 0.03 |
| Trimethylglycine | 1 |
| Sodium polyaspartate | 0.1 |
| Ascorbic acid dl-α-tocopherol phosphoric | 0.1 |
| acid diester potassium salt | |
| Thiotaurine | 0.1 |
| Green tea extract | 0.1 |
| Mentha Piperita (Peppermint) leaf extract | 0.1 |
| Iris Florentina Root extract | 1 |
| EDTA 3Na | 0.1 |
| Carboxyvinyl polymer | 0.05 |
| Precisely synthesized sodium polyacrylate | 0.01 |
| (Synthesis Example 1) | |
| Potassium hydroxide | 0.02 |
| Phenoxyethanol | Suitable amount |
| Perfume | Suitable amount |
| Purified water | Balance |
| | Total: 100.0 |

### Formulation Example 2: Cosmetic Lotion

<Formulation>

| | Blending amount |
|---|---|
| Component | (% by mass) |
| Dipropylene glycol | 5 |
| Glycerin | 6 |
| Butylene glycol | 7 |
| Polyethylene glycol | 5 |
| Methyl gluceth-10 | 2 |
| Triethylhexanoin | 0.3 |
| PEG-60 hydrogenated castor oil | 0.5 |
| Polyglyceryl-2 diisostearate | 0.4 |
| Carboxyvinyl polymer | 0.05 |
| Precisely synthesized sodium | 0.05 |
| polyacrylate (Synthesis Example 1) | |
| Potassium hydroxide | 0.02 |
| Tranexamic acid | 1 |
| Sodium alginate | 0.1 |
| Citric acid | Suitable amount |
| Phenoxyethanol | Suitable amount |
| Sodium metaphosphate | Suitable amount |
| Perfume | Suitable amount |
| Purified water | Balance |
| | Total: 100.0 |

### Formulation Example 3: Cosmetic Lotion

<Formulation>

| | Blending amount |
|---|---|
| Component | (% by mass) |
| Glycerin | 2 |
| 1,3-Butylene glycol | 4 |
| Erythritol | 1 |
| Polyoxyethylene methyl glucoside | 1 |
| Polyoxyethylene hydrogenated castor oil | 0.5 |
| Dimethyl acrylamide | |
| Crosslinked sodium N,N-dimethylacrylamide-2- | |
| acrylamido-2-methylpropanesulfonic acid copolymer | |
| Precisely synthesized sodium polyacrylate | 0.03 |
| (Synthesis Example 2) | |
| N-coconut oil fatty acid acyl-L-arginine-ethyl DL- | 0.1 |
| pyrrolidonecarboxylic acid | |
| Citric acid | 0.02 |
| Sodium citrate | 0.08 |
| Phenoxyethanol | Suitable amount |
| Purified water | Balance |
| | Total: 100.0 |

### Formulation Example 4: Cosmetic Lotion

<Formulation>

| | Blending amount |
|---|---|
| Component | (% by mass) |
| Glycerin | 10 |
| Ethanol | 5 |
| Dipropylene glycol | 6 |
| PEG/PPG-14/7 dimethyl ether | 1 |
| PPG-13 decyltetradeceth-24 | 0.5 |
| (Acryloyldimethylltaurate/VP) copolymer | 0.05 |
| Precisely synthesized sodium polyacrylate | 0.03 |
| (Synthesis Example 3) | |
| Lactic acid | 1 |
| Tranexamic acid | 2 |
| Xanthan gum | 0.1 |
| Citric acid | Suitable amount |
| Sodium citrate | Suitable amount |
| Phenoxyethanol | Suitable amount |
| Disodium | edetate Suitable amount |
| Perfume | Suitable amount |
| Purified water | Balance |
| | Total: 100.0 |

### Formulation Example 5: Cosmetic Lotion

<Formulation>

| | Blending amount |
|---|---|
| Component | (% by mass) |
| Mineral oil | 0.5 |
| Isostearyl alcohol | 0.5 |
| Glycerin | 4 |
| Dipropylene glycol | 5 |
| Butylene glycol | 5 |
| Polyethylene glycol | 4 |
| Isostearic acid | 1 |
| PEG/PPG-14/7 dimethyl ether | 5 |
| Sorbitan sesquiisostearate | 0.3 |
| PEG-30 soy sterol | 1 |
| (Acryloyldimethyltaurate/beheneth-25 | 0.05 |
| methacrylate) copolymer | |
| Precisely synthesized sodium polyacrylate | 0.05 |
| (Synthesis Example 3) | |
| Tranexamic acid | 1 |
| Dipotassium glycyrrhizinate | 0.05 |
| Citric acid | Suitable amount |
| Sodium metaphosphate | Suitable amount |
| Phenoxyethanol | Suitable amount |
| Perfume | Suitable amount |
| Purified water | Balance |
| | Total: 100.0 |

### Formulation Example 6: Whitening Emulsion

<Formulation>

| | Blending amount |
|---|---|
| Component | (% by mass) |
| Hydrogenated polydecene | 1 |
| Dimethicone | 1 |
| Cyclomethicone | 2 |
| Behenyl alcohol | 0.2 |
| Batyl alcohol | 0.1 |
| Glycerin | 7 |
| Butylene glycol | 8 |
| Cetyl ethylhexanoate | 2 |
| Polysorbate 60 | 0.1 |
| PEG-60 hydrogenated castor oil | 0.1 |
| Ascorbyl glucoside | 2 |
| Xanthan gum | 0.05 |
| Sodium polyacrylate/acryloyldimethyl taurate | 2.5 |
| copolymer dispersion (actual content) | |
| Acrylic acid/alkyl methacrylate copolymer | 0.1 |
| Precisely synthesized sodium polyacrylate | 0.5 |
| (Synthesis Example 1) | |
| Disodium edetate | Suitable amount |
| Potassium hydroxide | Suitable amount |
| Citric acid | Suitable amount |
| Sodium metaphosphate | Suitable amount |
| Phenoxyethanol | Suitable amount |
| Iron oxide | Suitable amount |
| Purified water | Balance |
| | Total: 100.0 |

### Formulation Example 7: Emulsion

<Formulation>

| | Blending amount |
|---|---|
| Component | (% by mass) |
| Carboxydecyl trisiloxane | 1 |
| Dimethicone | 1.5 |
| Cyclomethicone | 2.5 |
| Diphenylsiloxy phenyl trimethicone | 1 |
| Behenyl alcohol | 1 |
| Batyl alcohol | 1 |
| Glycerin | 2 |
| Dipropylene glycol | 7 |
| Glyceryl stearate (SE) | 1 |
| PEG-5 glyceryl stearate | 0.5 |
| Tranexamic acid | 2 |
| Carbomer | 0.1 |
| Precisely synthesized sodium polyacrylate | 0.3 |
| (Synthesis Example 2) | |
| Potassium hydroxide | Suitable amount |
| Sodium metaphosphate | Suitable amount |
| Iron oxide | Suitable amount |
| Phenoxyethanol | Suitable amount |
| Disodium edetate | Suitable amount |
| Perfume | Suitable amount |
| Purified water | Balance |
| | Total: 100.0 |

### Formulation Example 8: Emulsion

<Formulation>

| | Blending amount |
|---|---|
| Component | (% by mass) |
| Hydrogenated polydecene | 2 |
| Vaseline | 1 |
| Dimethicone | 1 |
| Ethanol | 2 |
| Behenyl alcohol | 1.2 |
| Glycerin | 8 |
| Butylene glycol | 4 |
| Stearic acid | 0.5 |
| Behenic acid | 0.5 |
| Ceteth-25 | 0.3 |
| Sodium stearoyl glutamate | 0.3 |
| Crosslinked sodium N,N-dimethylacrylamide-2- | 0.5 |
| acrylamido-2-methylpropanesulfonic acid copolymer | |
| Precisely synthesized sodium polyacrylate | 0.8 |
| (Synthesis Example 2) | |
| 4-Methoxysalicylic acid potassium salt | 1 |
| Tranexamic acid | 2 |
| Tocopherol acetate | 1 |
| Potassium hydroxide | Suitable amount |
| Phenoxyethanol | Suitable amount |
| Disodium edetate | Suitable amount |
| Perfume | Suitable amount |
| Purified water | Balance |
| | Total: 100.0 |

### Formulation Example 9: Emulsion

<Formulation>

| | Blending amount |
|---|---|
| Component | (% by mass) |
| Vaseline | 5 |
| Behenyl alcohol | 0.5 |
| Batyl alcohol | 0.5 |
| Glycerin | 7 |
| 1,3-Butylene glycol | 7 |
| 1,2-Pentanediol | 1 |
| Xylit | 3 |
| Polyethylene glycol 20000 | 2 |
| Hydrogenated oil | 2 |
| Jojoba oil | 2 |
| Squalane | 5 |
| Isostearic acid | 0.5 |
| Pentaerythrityl tetra-2-ethylhexanoate | 2 |
| Polyoxyethylene hydrogenated castor oil | 0.5 |
| Betaine lauryldimethylaminoacetate | 0.4 |
| Sodium pyrosulfite | 0.01 |
| Sodium hexametaphosphate | 0.05 |
| Dipotassium glycyrrhizinate | 0.05 |
| Trimethylglycine | 3 |
| Arbutin | 3 |
| Yeast extract | 0.1 |
| Tocopherol acetate | 0.1 |
| Thiotaurine | 0.1 |
| Sophora Angustifolia Root extract | 0.1 |
| Pyrus Cydonia Seed extract | 0.1 |
| Carboxyvinyl polymer | 0.2 |
| Precisely synthesized sodium polyacrylate | 0.3 |
| (Synthesis Example 3) | |
| Sodium hydroxide | Suitable amount |
| Phenoxyethanol | Suitable amount |
| Red iron oxide | Suitable amount |
| Purified water | Balance |
| | Total: 100.0 |

### Formulation Example 10: Emulsion

<Formulation>

| | Blending amount |
|---|---|
| Component | (% by mass) |
| Dimethyl polysiloxane | 3 |
| Decamethylcyclopentasiloxane | 4 |
| Ethanol | 5 |
| Glycerin | 6 |
| 1,3-Butylene glycol | 5 |
| Polyoxyethylene methyl glucoside | 3 |
| Sunflower oil | 1 |
| Squalane | 2 |
| Potassium hydroxide | 0.1 |
| Sodium hexametaphosphate | 0.05 |
| Hydroxypropyl-β-cyclodextrin | 0.1 |
| Dipotassium glycyrrhizinate | 0.05 |
| Eriobotrya Japonica Leaf extract | 0.1 |
| L-sodium glutamate | 0.05 |
| Fennel extract | 0.1 |
| Yeast extract | 0.1 |
| Lavender oil | 0.1 |
| Rehmannia Root extract | 0.1 |
| Dimorpholinopyridazinone | 0.1 |
| Xanthan gum | 0.1 |
| Carboxyvinyl polymer | 0.1 |
| Acrylic acid/alkyl methacrylate copolymer | 0.1 |
| Precisely synthesized sodium polyacrylate | 0.5 |
| (Synthesis Example 1) | |
| Red iron oxide | Suitable amount |
| Yellow iron oxide | Suitable amount |
| Paraben | Suitable amount |
| Purified water | Balance |
| | Total: 100.0 |

### Formulation Example 11: Emulsion

<Formulation>

| | Blending amount |
|---|---|
| Component | (% by mass) |
| Dimethicone | 2 |
| Ethanol | 3 |
| Behenyl alcohol | 1 |
| Glycerin | 3 |
| Dipropylene glycol | 5 |
| Butylene glycol | 3 |
| Triethylhexanoin | 1 |
| Sodium methyl stearoyl taurate | 0.1 |
| Citric acid | 0.18 |
| Sodium citrate | 0.02 |
| Tranexamic acid | 2 |
| Xanthan gum | 0.05 |
| (Acryloyldimethyltaurate/beheneth-25 methacrylate) | 0.5 |
| copolymer | |
| Precisely synthesized sodium polyacrylate | 0.5 |
| (Synthesis Example 1) | |
| Phenoxyethanol | Suitable amount |
| Disodium edetate | Suitable amount |
| Perfume | Suitable amount |
| Purified water | Balance |
| | Total: 100.0 |

### Formulation Example 12: Emulsion

<Formulation>

| | Blending amount |
|---|---|
| Component | (% by mass) |
| Dimethyl polysiloxane | 3 |
| Methylphenyl polysiloxane | 3 |
| Ethanol | 5 |
| Glycerin | 4 |
| Dipropylene glycol | 5 |
| 1,3-Butylene glycol | 5 |
| Di-2-ethylhexyl succinate | 3.5 |
| Potassium hydroxide | 0.1 |
| Sodium hexametaphosphate | 0.1 |
| Thiotaurine | 0.1 |
| Edetate trisodium | 0.1 |
| 4-t-butyl-4′-methoxydibenzoylemethane | 3 |
| 2-Ethylhexyl p-methoxycinnamate | 3 |
| Iron oxide | 0.01 |
| Acrylic acid/alkyl methacrylate copolymer | 0.1 |
| (Acryloyldimethylltaurate/VP) copolymer | 0.05 |
| Precisely synthesized sodium polyacrylate | 0.5 |
| (Synthesis Example 2) | |
| Paraben | Suitable amount |
| Perfume | Suitable amount |
| Purified water | Balance |
| | Total: 100.0 |

### Formulation Example 13: Gel

<Formulation>

| | Blending amount |
|---|---|
| Component | (% by mass) |
| Dimethyl polysiloxane | 5 |
| Glycerin | 2 |
| 1,3-Butylene glycol | 5 |
| Polyethylene glycol 1500 | 3 |
| Polyethylene glycol 20000 | 3 |
| Cetyl octanoate | 3 |
| Citric acid | 0.01 |
| Sodium citrate | 0.1 |
| Sodium hexametaphosphate | 0.1 |
| Dipotassium glycyrrhizinate | 0.1 |
| Ascorbyl glucoside | 2 |
| Tocopherol acetate | 0.1 |
| Scutellaria Baicalensis extract | 0.1 |
| Saxifraga Sarmentosa extract | 0.1 |
| Edetate trisodium | 0.1 |
| Xanthan gum | 0.3 |
| Acrylic acid/alkyl methacrylate copolymer | 0.05 |
| Agar powder | 1.5 |
| Precisely synthesized sodium polyacrylate | 0.2 |
| (Synthesis Example 1) | |
| Phenoxyethanol | Suitable amount |
| Dibutyl hydroxytoluene | Suitable amount |
| Purified water | Balance |
| | Total: 100.0 |

### <Production Method>

A translucent emulsion composition was produced by an ordinary method and cooled to 30° C. or lower to be gelated. When the gel was sufficiently solidified, the gel was crushed by a disper to give a microgel (having an average particle size of 70 μm). Then the microgel was degassed to give a gel-like product.

### Formulation Example 14: Beauty Serum

<Formulation>

| | Blending amount |
|---|---|
| Component | (% by mass) |
| Ethanol | 10 |
| PPG-13 decyltetradeceth-24 | 0.1 |
| Salicylic acid | 0.5 |
| Tranexamic acid | 1 |
| Xanthan gum | 0.2 |
| Gellan gum | 0.5 |
| Sodium chloride | 0.9 |
| Potassium hydroxide | Suitable amount |
| Sodium pyrosulfite | Suitable amount |
| Disodium edetate | Suitable amount |
| Purified water | Balance |
| | Total: 100.0 |

1. A cosmetic, comprising:
components (a) and (b) wherein;
component (a) is polyacrylic acid or a salt thereof or poly(2-acrylamido-2-methylpropanesulfonic acid) or a salt thereof that has a weight-average molecular weight of 500,000 to 8,000,000, is linear, and has a thread length of 10 mm or less at room temperature when formed into a 1% by mass solution, wherein the thread length is a distance determined by: putting a round disk having a diameter of about 1 cm uniformly and lightly into contact with a surface of the solution; lowering a container that contains the solution at a velocity of 5 mm/sec, and measuring the distance that the container descended until a thread of the solution is cut; and
component (b) is a crosslinked water-swellable polymer having a crosslinking density of 0.01 to 1% by mole, or a microgel obtained by pulverizing a gel consisting of a hydrophilic compound having gelating ability.

2. The cosmetic, according to claim 1, wherein:
the crosslinked water-swellable polymer of the component (b) is one or more selected from the group consisting of carboxyvinyl polymer, acrylamidealkylsulfonic acid/beheneth-25 crosslinked copolymer, acrylamidealkyl sulfonic acid/vinylpyrrolidone crosslinked copolymer, and acrylamidealkylsulfonic acid/alkylacrylamide crosslinked copolymer.

3. The cosmetic, according to claim 1, wherein:
the microgel of the component (b) is a hydrophilic polysaccharide.

4. The cosmetic, according claim 1, wherein:
a content of the compound having a molecular weight of 10,000,000 or more in the component (a) is 10% by mass or less.

5. The cosmetic, according to claim 2, wherein:
the microgel of the component (b) is a hydrophilic polysaccharide.

6. The cosmetic, according to claim 2, wherein:
the content of the compound having the molecular weight of 10,000,000 or more in the component (a) is 10% by mass or less.

7. The cosmetic, according to claim 3, wherein:
the content of the compound having the molecular weight of 10,000,000 or more in the component (a) is 10% by mass or less.
